(19) United States
(12) Patent Application Publication
(10) Pub. No.: US 2005/0069960 A1
(43) Pub. Date: Mar. 31, 2005

(54) Novel complementing receptor-ligand pairs and adoptive immunotherapy using the same

(76) Inventor: Charles A. Nicolette, Framingham, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 10/993,841

(22) Filed: Nov. 19, 2004

Related U.S. Application Data
(63) Continuation of application No. 09/663,075, filed on Sep. 15, 2000, now abandoned.
Application No. 09/663,075 is a continuation of application No. PCT/US99/06022, filed on Mar. 19, 1999.
(60) Provisional application No. 60/078,907, filed on Mar. 20, 1998.

(51) Int. Cl. G01N 33/53; G01N 33/567; A61K 48/00

(52) U.S. Cl. 435/7.2; 514/44

(57) ABSTRACT

This invention provides a screen to identify novel therapeutic receptor-ligand pairs. In one embodiment, the receptor-ligand pairs identified by this method induce proliferation of tumor-infiltrating lymphocytes without systemic toxicity associated with the administration of wild-type cytokines. Diagnostic and therapeutic methods using the cytokine-receptor pairs identified by this screen also are provided.

### CROSS-REFERENCE TO RELATED APPLICATION

This application claims priority under 35 U.S.C. § 119(e) of U.S. Provisional Application No. 60/078,907, filed Mar. 20, 1998, the contents of which are hereby incorporated by reference into the present disclosure.

### TECHNICAL FIELD

This invention is in the field of molecular immunology and medicine. In particular, this invention is directed to identifying novel receptor-ligand pairs and use of the pairs for immunotherapy.

### BACKGROUND

Immunotherapy of cancer has traditionally been categorized as active (e.g., cancer vaccines), passive (e.g., adoptive cellular therapy or monoclonal antibody therapy), and non-specific (e.g., cytokine therapies). These therapies exploit the discovery that antitumor immune responses occur and can be identified. Genes coding for tumor-associated antigens yielding peptides recognized by antitumor-specific cytotoxic T-lymphocytes (CTLs) have been cloned and characterized. Beyond CTLs, different effector and accessory cells, including NK cells, eosinophils, T helper lymphocytes, macrophages, and dendritic cells are believed to cooperate to generate an effective immune response.

Cytokines are important components of all anti-cancer therapies. Tumor-specific cell surface antigens distinguish tumor cells from normal cells; however, some tumor cells are deficient in intracellular processes required for antigen presentation to T cells. Cytokines can compensate for many and perhaps all the defects in tumor antigen presentation and can amplify the immune response to tumors by both antigen specific and antigen nonspecific cells. The mechanisms by which cytokines are able to elicit an immune response are in some cases quite complicated and multifactorial (e.g., interleukin-2 (“IL-2”)), in other cases seemingly more straightforward (e.g., GM-CSF), and in yet other cases undefined (IL-1, IL-7). However, it is clear that the local production of a cytokine is a key component to successful therapy.

Direct delivery of cytokines such as IL-2, is believed to have a direct effect on tumor-specific CTLs, by activating precursors and/or reactivating anergized CTLs. They also have been shown to play a major role in mediating differentiation, proliferation, and/or activation of the various partner cells.

Gene therapy with cytokine-expressing cells is another promising use of cytokines for cancer therapy. Mouse tumor cells engineered to express certain cytokines, particularly IL-2, are rejected and often vaccinate the mice against a subsequent challenge with non-engineered tumor cells. Bubenik et al. (1990) Immunol. Lett. 23:287; Fearon et al. (1990) Cell 60:397; and Gansbacher et al. (1990) J. Exp. Med. 172:1217. Numerous studies have been conducted with other cytokines including IL-2 (Cavallo et al. (1992) J. Immunol. 149:3627-35); IL-4 (Pericle et al. (1994) J. Immunol. 153:5659-73); IL-6 (Allione et al. (1994) Cancer Res. 54:6022-6); IL-7 (Musiani et al. (1996) Lab. Invest. 74:146-57); IL-10 (Giovarelli et al. (1995) J. Immunol. 155:3112-23); GM-CSFs (Allione (1994) supra.); interferon alpha (IFN α) (Ferrantini et al. (1994) J. Immunol. 153:460-4-15); IFN γ (Lollini et al. (1993) Int. J. Cancer 55:320-9); and tumor necrosis factor (TNF)-α (Allione et al. (1994) supra).

One of the first attempts at adoptive immunotherapy involved the isolation of tumor infiltration lymphocytes (TIL) from surgically resected tumors, expansion ex vivo, and re-infusion into the patient. Early studies on TIL infusions in mouse models suggested that the ability of TILs to proliferate in vivo was a necessary adjunct to clinical response (i.e., tumor regression). Co-administration of interleukin-2 (IL-2) achieved this result.

The recruitment of host CD8⁺ T cells to tumor sites by adoptively transferred TIL+recombinant IL-2 (“rIL-2”) has been shown to be required for effective tumor eradication in mouse model systems (Burger et al. (1995) Surgery 117:325-333). In some human patients, adoptive immunotherapy with TIL and rIL-2 results in dramatic regressions in patients with metastatic melanoma and renal cell carcinoma However, the severe cardiovascular and hemodynamic toxic effects of IL-2 are limiting factors for this therapy.

In cultures of IL-2 dependent cells, the rate of consumption of exogenously added IL-2 is proportional to the number of cells in culture expressing IL-2 receptors. Studies on the adoptive transfer of LAK cells in a mouse model system demonstrated that the minimum dose of IL-2 needed to maintain antitumor activity of LAK cells in vivo is 150,000 U to 250,000 U/kg twice or thrice daily for 6 days (Lotze et al. (1981) Cancer Res. 41:4420). In humans, however, the maximum tolerable dose of IL-2 when given systemically with LAK cells is 100,000 U/kg given thrice daily for 4 days (Rosenberg et al. (1985) N. Eng. J. Med. 313:1485). It can be reasonably postulated that the maximum tolerated dose in at least a subset of treated patients is subtherapeutic with respect to TIL proliferation.

Thus, it is clear that cytokines play many roles in successful cancer immunotherapies and immune homeostasis. However, when used in current therapies, their inherent toxicity is a limitation that needs to be addressed prior to wide-spread clinical use. Additionally, the mutifactorial effect of many cytokines produces deleterious effects which may be avoided by a targeted and localized expression of cytokines. This invention satisfies these needs and provides related advantages as well.

### DISCLOSURE OF THE INVENTION

This invention provides compositions and methods for the selective activation of receptors by providing mutant receptor-ligand pairs. In one respect, this invention enhances the benefits of cancer immunotherapy and minimizes the toxic side effects of adjuvant cytokine administration, e.g., IL-2 administration, by providing mutant cytokine receptor-ligand binding pairs that stimulate TIL without toxic systemic side effects. These methods can be achieved by co-administering a host cell expressing the mutant receptor and its mutant binding partner.

This invention also provides a screen to identify novel receptor-ligand pairs which are useful for therapy. The receptor-ligand pairs identified by this method are highly specific for each other and possess an inherently lower clearance rate because they are not utilized and therefore not internalized by cells expressing wild-type receptors. In one embodiment, the receptor-ligand pairs are cytokine receptor-ligand pairs. In this embodiment, the pair will induce proliferation of tumor-infiltrating lymphocytes without systemic toxicity associated with the administration of wild-type cytokines or alternatively, induce the proliferation of hematopoietic stem cells. These results are achieved because the ligand of the pair identified by this screen binds with higher affinity to the corresponding wild-type receptor or alternatively, the receptor is not activated by the corresponding wild-type ligand.

This invention also provides therapy by administering to a subject a polynucleotide encoding a novel mutated receptor identified by the above screen, either alone, or transduced into a host cell which is then administered to the subject. The mutated ligand, either as protein or as a polynucleotide encoding the protein, is then administered to the subject.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); the series “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987, and periodic updates); “PCR: The Polymerase Chain Reaction”, (Mullis et al., eds., 1994); “Current Protocols in Immunology” (J. E. Coligan et al., eds., 1991).

Definitions

As used in the specification and claims, the singular form “a”, “an” and “the” include plural references unless the context clearly dictates otherwise. For example, the term “a cell” includes a plurality of cells, including mixtures thereof.

A “ligand” is intended to include any substance that either inhibits or stimulates the activity of a receptor, e.g., a cytokine or an antibody. An “agonist” is defined as a ligand increasing the functional activity of a receptor (i.e. signal transduction through the receptor). An “antagonist” is defined as a ligand decreasing the functional activity of a receptor either by inhibiting the action of an agonist or by its own activity.

A “receptor” is intended to include any molecule present inside or on the surface of a cell, which molecule may effect cellular physiology when either inhibited or stimulated by a ligand. Typically, receptors which may be used for the present purpose comprise an extracellular domain with ligand-binding properties, a transmembrane domain which anchors the receptor in the cell membrane and a cytoplasmic domain which generates a cellular signal in response to ligand binding (“signal transduction”). In some cases, e.g. with adrenergic receptors, the transmembrane domain is in the form of up to several helical, predominantly hydrophobic structures spanning the cell membrane and part of the transmembrane domain has ligand-binding properties.

As used herein, the term “receptor-ligand pair” means a pair of biological molecules that have a specific affinity for each other. One member of the receptor-ligand pair must be localized on a surface of a membrane, and preferably on a surface of the plasma membrane, at some point in its in vivo existence. Preferably, the affinity arises by virtue of the members of the receptor-ligand pair possessing complementary three-dimensional structures, e.g., as seen in the relationship between an enzyme and its substrate. Within a given receptor-ligand pair, either member may be considered to be the ligand or the receptor. Examples of ligand-receptor pairs include all of the following: a cell surface receptor (e.g., a molecule that transmits a signal, e.g., across a cell membrane, when bound to its ligand) and its ligand, e.g., an oncogene-encoded receptor and its ligand or a growth factor and its receptor, e.g., a lymphokine and its receptor, e.g., an interleukin and its receptor; an enzyme and its substrate; an enzyme and a specific inhibitor or other non-catalyzable substrate of the enzyme; a hormone and its receptor; a first subunit of a multimeric protein and a second subunit of the multimeric protein, e.g., two subunits of an immunoglobin molecule; a polypeptide portion of a protein and a non-peptide cofactor of the protein; a molecule involved in cellular adhesion (e.g., a carbohydrate involved in cell adhesion; a cadherin; a cell adhesion molecule (CAM), e.g., cell-CAM, neural N-CAM, or muscle N-CAM; a laminin; a fibronectin; or an integrin) and the molecule to which it binds, which may or may not be a cellular adhesion molecule; a first component of an organelle, the mitotic or meiotic apparatuses, or other subcellular structure, that displays a specific interaction with a second component of the same structure or a related structure; a lectin and a carbohydrate; a toxin and its receptor, e.g., diphtheria toxin and its cell surface receptor; a component of a virus and its cell surface receptor; or, an IgE molecule and an IgE receptor, e.g., the IgE receptor found on mast cells, or any other Ig molecule and its receptor (where receptor does not include the antigen against which the antibody molecule is directed, i.e., an antibody and its antigen are not within the definition of a receptor and its ligand, as used herein). A first strand of nucleic acid and a second strand complementary to the first are not within the definition of a ligand and its receptor.

Specific binding pair, as used herein, means any pair of molecules, including a first and a second member, which have a specific affinity for each other. Examples of specific binding pairs include ligands and receptors, as defined above, avidin and biotin, and antibodies and their antigens.

“Affinity” is used to describe the strength of binding of a ligand to its receptor, e.g., an antibody to its antigen. The affinity can be measured as the ratio of receptor-ligand complex to free reactants at equilibrium, and the affinity constant is equivalent to the association constant of the binding of a monvalent ligand to one binding site on the antibody. For antibody-antigen interactions, it should therefore be distinguished from the avidity of an antibody for its antigen, which is the measure of overall strength of binding of an antigen to antibody taking into account the increased strength of binding when the antigen and antibody are multivalent. The affinity of a antibody for a monovalent hapten can be determined from a scatchard plot of equilibrium binding experiments. For monoclonal antibodies where only one class of binding site is present, the slope of linear Scatchard plot is the negative of the affinity constant, which his the reciprocal of K_{d} the equilibrium binding (dissociation) constant for the antigen-antibody interaction. For a population of antibodies of slightly different affinities for their cognate antigen, a curved Schatchard plot is obtained and the average affinity is calculated.

“Mutant” refers to an alteration of the primary sequence of a receptor such that it differs from the wild type or naturally occurring sequence.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1α), interleukin-11 (IL-11), MIP-1α, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the invention.

“Co-stimulatory molecules” are involved in the interaction between receptor-ligand pairs expressed on the surface of antigen presenting cells and T cells. Research accumulated over the past several years has demonstrated convincingly that resting T cells require at least two signals for induction of cytokine gene expression and proliferation (Schwartz R. H. (1990) Science 248:1349-1356 and Jenkins M. K. (1992) Immunol. Today 13:69-73). One signal, the one that confers specificity, can be produced by interaction of the TCR/CD3 complex with an appropriate MHC/peptide complex. The second signal is not antigen specific and is termed the “co-stimulatory” signal. This signal was originally defined as an activity provided by bone-marrow-derived accessory cells such as macrophages and dendritic cells, the so called “professional” APCs. Several molecules have been shown to enhance co-stimulatory activity. These are heat stable antigen (HSA) (Liu Y. et al. (1992) J. Exp. Med. 175:437-445); chondroitin sulfate-modified MHC invariant chain (Ii-CS) (Naujokas M. F. et al. (1993) Cell 74:257-268); intracellular adhesion molecule 1 (ICAM-1) (Van Seventer G. A. (1990) J. Immunol. 144:4579-4586); B7-1 and B7-2/B70 (Schwartz R. H. (1992) Cell 71:1065-1068). Other important co-stimulatory molecules are CD40, CD54, CD80, CD86. Also encompassed by the term “co-stimulatory molecule” are any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter. It is intended, although not always explicitly stated, that molecules having similar biological activity as wild-type or purified co-stimulatory molecules (e.g., recombinantly produced or muteins thereof) are intended to be used within the spirit and scope of the invention.

“Lymphocytes” as used herein, are spherical cells with a large round nucleus (which may be indented) and scanty cytoplasm. They are cells that specifically recognize and respond to non-self antigens, and are responsible for development of specific immunity. Included within “lymphocytes” are B-lymphocytes and T-lymphocytes of various classes.

“Cytotoxic T lymphocytes” or “CTLs” are T cells which bear the CD3 cell surface determinant and mediate the lysis of target cells bearing cognate antigens. CTLs may be of either the CD8<+> or CD4<+> phenotype. CTLs are generally antigen-specific and MHC-restricted in that they recognize antigenic peptides only in association with the major histocompatibility complex (MHC) molecules on the surface of target cells. CTLs may be specific for a wide range of viral, tumor or allospecific antigens, including HIV, EBV, CMV and a wide range of tumor antigens. Growth or proliferation may be measured, for example, by any in vitro proliferation or growth assay or by any assay measuring the ability of the CTL to persist in vivo. Specific examples of suitable assays are known in the art and disclosed U.S. Pat. No. 5,747,292. CTLs capable of enhanced growth or viability may have augmented ability to destroy target cells bearing the foreign antigens or provide long-term immunologic memory.

The term “genetically modified” means containing and/or expressing a foreign gene or nucleic acid sequence which in turn, modifies the genotype or phenotype of the cell or its progeny. In other words, it refers to any addition, deletion or disruption to a cell's endogenous nucleotides.

A “gene delivery vehicle” is defined as any molecule that can carry inserted polynucleotides into a host cell. Examples of gene delivery vehicles are liposomes, biocompatible polymers, including natural polymers and synthetic polymers, lipoproteins, polypeptides, polysaccharides, lipopolysaccharides, artificial viral envelopes, metal particles, and bacteria, viruses, such as baculovirus, adenovirus, adeno-associated virus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Vector” means a self-replicating nucleic acid molecule that transfers an inserted nucleic acid molecule into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above functions.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“PCR primers” refer to primers used in “polymerase chain reaction” or “PCR,” a method for amplifying a DNA base sequence using a heat-stable polymerase such as Taq polymerase, and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce exponential and highly specific amplification of the desired sequence. (See, e.g., “PCR 2: A Practical Approach” supra). PCR also can be used to detect the existence of the defined sequence in a DNA sample.

“Host cell” or “recipient cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be procaryotic or eucaryotic, and include but are not limited to bacterial cells, yeast cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

The term “culturing” refers to the in vitro propagation of cells or organisms on or in media of various kinds. It is understood that the descendants of a cell grown in culture may not be completely identical (either morphologically, genetically, or phenotypically) to the parent cell. By “expanded” is meant any proliferation or division of cells.

A “subject” is a vertebrate, preferably a mammal, more preferably a human. Mammals include, but are not limited to, murines, simians, humans, farm animals, sport animals, and pets.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester ether etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

This invention provides a method for identifying novel receptor-ligand binding pairs by contacting a cell expressing a mutated receptor with a putative ligand and assaying for receptor-ligand binding and biological response. The receptor-ligand pair identified by the screen will bind with higher affinity to each other as compared to the corresponding wild-type receptor-ligand pair. Alternatively, the pair will bind with lower or no affinity for its mate, for example, mutated receptor should possess little or no affinity for wild-type ligand and/or mutated ligand should possess little or no affinity for wild-type receptor. In one aspect of this invention, a mutated receptor or ligand binds with at least 10 fold less, and more preferably, at least 20 fold less, affinity for its corresponding wild-type ligand or receptor. The mutated receptor-ligand pair also provides the biological response of the corresponding wild-type pair.

As is apparent to those of skill in the art, one may practice the invention by first providing a mutant ligand and then providing a cell expressing a putative mutated receptor. Receptor-ligand binding and biological response is then assayed.

Any protein that has a toxic profile and that is produced in a pathway involving receptor-ligand pairs is intended to be encompassed by this invention. Non-limiting examples include the receptor-ligand pairs of IL-2, TNF-α, GM-CSF, and IFN-α and γ.

To perform the screen, putative ligand-binding pairs are constructed and first assayed for affinity as compared to wild-type receptor-ligand pairs. Any known method can be used to satisfy this aspect of the invention. For the purpose of illustration only, Applicant has described the yeast two-hybrid screen as an embodiment for the practice of this invention.

This invention also provides the isolated mutant receptor and mutated ligand identified by the above screen, as well as polynucleotides encoding them. Host cells, such as tumor infiltrating lymphocytes, dendritic cells, tumor cells or hematopoietic stem cells, transduced with the polynucleotides also are encompassed by this invention. Compositions, especially pharmaceutical compositions comprising the mutated receptor, ligand, encoding polynucleotides and transduced host cells, alone or in combination with each other, are further provided herein.

The receptor-ligand pairs are useful to selectively activate a biological response and/or to avoid toxic side effects associated with traditional therapies, e.g., IL-2 therapy. In one aspect, an effective amount of a host cell expressing a polynucleotide expressing and presenting the mutated receptor is administered to the subject. An effective amount of a mutated ligand that binds to the receptor with a higher affinity as compared to the wild-type receptor-ligand binding pair is administered either prior to, concurrently or subsequently to administration of the host cell to activate the biological response mediated by the binding of the corresponding wild-type receptor-ligand pair. The compositions can be used in conjunction with cancer vaccines to induce an immune response in the subject thereby reducing tumor burden and treating cancer.

This method can be further modified by administering an effective amount of a co-stimulatory molecule to the subject. The molecule is administered either as a protein or in the form of a polynucleotide encoding the protein.

In a further aspect of this invention, the mutated receptor-ligand pairs also can be used to induce an immune response in a subject by administering, in situ, an effective amount of a polynucleotide encoding a mutated receptor to a tumor, preferably in a viral vector. The viral vector is directly injected into the tumor to transduce (in vivo) tumor-infiltrating lymphocytes in the tumor in situ. Subsequently (e.g., a period of several days) the mutated ligand of the pair is administered. The mutated ligand may be administered in the form of a polynucleotide encoding the ligand or transduced in a TIL which is then administered to the subject. This method can be further modified by administering an effective amount of a co-stimulatory molecule to the subject.

As is apparent to one of skill in the art, polynucleotides as disclosed herein are suitably transduced using naked DNA using a suitable gene delivery vechicle.

The following examples are intended to illustrate and not limit the invention.

Polynucleotides, Proteins and Compositions

In one aspect, this invention provides a screen to identify novel mutated or chimeric receptor-ligand pairs. Prior to conducting the screen, putative mutated or chimeric receptors and putative ligand binding partners and the polynucleotides encoding the binding pairs are isolated and sequenced. The mutated and chimeric receptors and ligands can be produced from previously characterized receptor/ligand pairs, for example, the cytokine IL-2 has been previously characterized and shown to have potent anti-tumor effects. Several examples of such are provided below. IL-2 is particularly suited for use in the method of this invention since it has been determined that intratumoral delivery in animals of replication-deficient adenovirus vector expressing the murine IL-2 gene completely eradicated murine mastocytoma tumors in up to 75% of cases. Cordier et al. (1995) Gene Therapy 2:16-21. In a further study, this result was shown to be mostly due to nonspecific effectors. Levraud et al. (1997) J. Immunol. 158:3335-3343. IL-2 also has been shown to induce antitumoral immunity in mice. Haddada et al. (1993) Human Gene Therapy 4:703-711.

When the receptor of the pair is the IL-2 receptor, various specific embodiments are intended (see Tables 2 and 3, below). In one aspect a chimeric receptor complex consists of the IL2-γ subunit alone or in combination with either the IL-2 α or the IL-2 β subunits. Alternatively, the primary, secondary or tertiary structure of any of the subunits is modified from wild-type receptor or the ligand is modified, e.g., one of the previously characterized human IL-2 mutants listed in Table 2 and Table 3 below.

Other cytokines have been shown to possess anti-tumor activity and therefore mutants and receptors that specifically bind these growth factors can be screened using this method. Strong anti-tumor reactions have been shown to be elicited by numerous locally injected cytokines (IL-1, IL-2, IL4, IFN-gamma, G-CSF) and by cytokines released by engineered tumor cells (IL-1, IL-2, IL-3, IL-4, IL-5, IL-6, IL-7, IL-8, IL-10, IL-12, G-CSF, GM-CSF, IFN-alpha and IFN-gamma). Reviewed by Modesti et al. “Cytokine Dependent Tumor Recognition” in CYTOKINE INDUCED TUMOR IMMUNOGENICITY Forni et al. eds. (1994) (Academic Press, San Diego, Calif.). The peptide sequence and coding sequence for these cytokines and their receptors are known in the art and disclosed for example in GenBank under Accession Nos. J00264 (IL-2); g186336 (ILA); g181149 (GM-CSF); and g339739 (TNF-α).

The amount of cytokine produced by engineered cells (approximately 1×10⁵ cells/well) after 48 hours of culture in 1 mL of medium can be used to evaluate cytokine production by use of enzyme-linked immunoassay kits that are specific for individual cytokines (IL-6, IL-10, GM-CSF, and TNF-α (Endogen Inc., Boston, Mass.) and IL-12 (L. Adorini, Hoffmann-La Roche at the Istituto S. Raffaele, Milan]) or a biologic assay (IL-2 (Cavallo et al. (1992) supra), IL4 (Pericle et al. (1994) supra), IL-7 (Allione et al. (1994) supra), IFN-α (Ferrantini et al. (1994) supra), or IFN-γ (Lollini et al. (1993) supra)). When available, a standard of a known amount of each cytokine can be included in these assays to express the data as units per milliliter of cytokine produced. A representative clone can be selected that releases the above-specified conditions the amount of cytokine (IL-2, 3600 U/mL; IL-4, 40 U/mL; IL-6, 1250 U/mL; IL-7, 30 U/mL; IL-10, 620 U/mL; IL-12, 25 ng/mL; GM-CSF, 12 ng/mL; IFN α, 200 U/mL; IFN-γ, 6000 U/mL; TNF-α, 10 U/mL) that most efficaciously elicits an immune response to a subsequent challenge (Allione et al. (1994) supra and Musiani et al. (1996) supra). When mutants of IL-2 are screened, kinetic analysis of IL-2 on an IL-2/diptheria toxin may be utilized to analyze biological activity of mutant receptor-ligand pairs. Walz et al. (1990) Transplantation 49:198-201.

In another aspect of this invention, polynucleotides encoding mutant receptors are transduced into hematopoietic stem cells (pluripotent stem cells that are CD34⁺) that are then administered with a mutant ligand pair to selectively enhance proliferation of the transduced stem cells (e.g., wherein the receptor/ligand pair is G-CSF). Transduction ex vivo or in vivo using retroviral vectors, described below, are preferred for insertion of exogenous polynucleotide into hematopoietic stem cells. Cell populations useful in this method include, and are not limited to, cell populations obtained from bone marrow, both adult and fetal, mobilized peripheral blood (MPB) and umbilical cord blood. The use of umbilical cord blood is discussed, for instance, in Issaragrishi et al. (1995) N. Engl. J. Med. 332:367-369. Initially, bone marrow cells can be obtained from a source of bone marrow, including but not limited to, ilium (e.g., from the hip bone via the iliac crest), tibia, femora, vertebrate, or other bone cavities. Other sources of stem cells include, but are not limited to, embryonic yolk sac, fetal liver, and fetal spleen. The methods can include further enrichment or purification procedures or steps for stem cell isolation by positive selection for other stem cell specific markers. Suitable positive stem cell markers include, but are not limited to, CD34⁺ and Thy-1⁺.

For isolation of bone marrow, an appropriate solution can be used to flush the bone, including, but not limited to, salt solution, conveniently supplemented with fetal calf serum (FCS) or other naturally occurring factors, in conjunction with an acceptable buffer at low concentration, generally from about 5-25 mM. Convenient buffers include, but are not limited to, HEPES, phosphate buffers and lactate buffers. Otherwise bone marrow can be aspirated from the bone in accordance with conventional techniques.

Preferably, the cell population is initially subject to negative selection techniques to remove those cells that express lineage specific markers and retain those cells which are lineage negative (“LIN⁻”). LIN⁻ cells generally refer to cells which lack markers such as those associated with T cells (such as CD2, 3, 4 and 8), B cells (such as CD10, 19 and 20), myeloid cells (such as CD14, 15, 16 and 33), natural killer (“NK”) cells (such as CD2, 16 and 56), RBC (such as glycophorin A), megakaryocytes (CD41), mast cells, eosinophils or basophils. Methods of negative selection are known in the art. The absence or low expression of such lineage specific markers is identified by the lack of binding of antibodies specific to the cell specific markers, useful in so-called “negative selection”. Preferably the lineage specific markers include, but are not limited to, at least one of CD2, CD14, CD15, CD16, CD19, CD20, CD38, HLA-DR and CD71; more preferably, at least CD14 and CD15.

Various techniques can be employed to separate the cells by initially removing cells of dedicated lineage. Monoclonal antibodies are particularly useful for identifying markers associated with particular cell lineages and/or stages of differentiation. The antibodies can be attached to a solid support to allow for crude separation. The separation techniques employed should maximize the retention of viability of the fraction to be collected. Various techniques of different efficacy can be employed to obtain “relatively crude” separations. Such separations are up to 10%, usually not more than about 5%, preferably not more than about 1%, of the total cells present not having the marker can remain with the cell population to be retained. The particular technique employed will depend upon efficiency of separation, associated cytotoxicity, ease and speed of performance, and necessity for sophisticated equipment and/or technical skill.

Procedures for separation can include, but are not limited to, physical separation, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of physical separation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342). These procedures are well known to those of skill in this art.

Techniques providing accurate separation include, but are not limited to, flow cytometry, which can have varying degrees of sophistication, e.g., a plurality of color channels, low angle and obtuse light scattering detecting channels, impedance channels, etc. Cells also can be selected by flow cytometry based on light scatter characteristics, where stem cells are selected based on low side scatter and low to medium forward scatter profiles. Cytospin preparations show the enriched stem cells to have a size between mature lymphoid cells and mature granulocytes.

The cells obtained as described above can be used immediately or frozen at liquid nitrogen temperatures and stored for long periods of time, being thawed and capable of being reused. The cells usually will be stored in 10% DMSO, 50% fetal calf serum (FCS), 40% RPMI 1640 medium. Once thawed, the cells can be expanded by use of growth factors

The mutated hematopoietic stem cell receptor may be co-administered with a therapeutic gene. Gene therapy using HSCs is useful to treat a genetic abnormality in lymphoid and myeloid cells that results generally in the production of a defective protein or abnormal levels of expression of the gene. For a number of these diseases, the introduction of a normal copy or functional homologue of the defective gene and the production of even small amounts of the missing gene product would have a beneficial effect. At the same time, overexpression of the gene product would not be expected to have deleterious effects. The following provides a non-exhaustive list of diseases for which gene transfer into HSCs is potentially useful. These diseases generally include bone marrow disorders, erythroid cell defects, metabolic disorders and the like. Hematopoietic stem cell gene therapy is beneficial for the treatment of genetic disorders of blood cells such as α and β-thalassemia, sickle cell anemia and hemophilia A and B in which the globin gene or clotting factor gene is defective. Another good example is the treatment of severe combined immunodeficiency disease (SCIDS), also known as the bubble boy syndrome, in which patients lack the adenosine deaminase (ADA) enzyme which helps eliminate certain byproducts that are toxic to T and B lymphocytes and render the patients defenseless against infection. Such patients are ideal candidates to receive gene therapy by introducing the ADA gene into their HSCs instead of the patient's lymphocytes as done in the past. Other diseases include chronic granulomatosis where the neutrophils express a defective cytochrome b and Gaucher disease resulting from an abnormal glucocerebrosidase gene product in macrophages.

The mutated receptor can be combined with additional transgenes to combat viral infections such as HIV and HTLV-1 infection. For example, HSCs can be genetically modified to render them resistant to infection by HIV. One approach is to inhibit viral gene expression specifically by using antisense RNA or by subverting existing viral regulatory pathways. Antisense RNAs complementary to retroviral RNAs have been shown to inhibit the replication of a number of retroviruses (To et al. (1986) Mol. Cell. Biol. 6:47584762; Rhodes and James (1991) AIDS 5:145-151; and von Reuden et al. (1991) J. Virol. 63:677-682).

Diseases other than those associated with hematopoietic cells can also be treated by genetic modification, where the disease is related to the lack of a particular secreted product including, but not limited to, hormones, enzymes, interferons, growth factors, or the like. By employing an appropriate regulatory initiation region, inducible production of the deficient protein can be achieved, so that production of the protein will parallel natural production, even though production will be in a different cell type from the cell type that normally produces such protein. It is also possible to insert a ribozyme, antisense or other message to inhibit particular gene products or susceptibility to diseases, particularly hematolymphotropic diseases.

The genes coding for the receptor and/or ligand can be cloned and sequenced using commercially available kits and technology. Gene delivery vehicles and/or host cells containing polynucleotides or nucleic acid sequences coding for these proteins and polypeptides also are within the scope of this invention. Recombinant methods for producing the mutated cytokine receptors and ligands are further provided, as well as the recombinantly produced proteins and polypeptides. Antibodies, including monoclonal antibodies, can be raised against these proteins and polypeptides using well known methods. Compositions containing any of the above noted genes, host cells, polypeptides, proteins or antibodies are further provided by this invention.

The proteins and polypeptides of this invention are obtainable by a number of processes well known to those of skill in the art, which include purification, chemical synthesis and recombinant methods. The receptor and ligand proteins and polypeptides identified by this invention can be purified from the transduced cell or tissue lysate using the process by methods such as immunoprecipitation with an appropriate antibody, and standard techniques such as gel filtration, ion-exchange, reversed-phase, and affinity chromatography. For such methodology, see for example Deutscher et al., “Guide To Protein Purification: Methods In Enzymology” (1990) Vol.182, Academic Press (San Diego, Calif.) and U.S. Pat. Nos. 5,707,798; 5,874,534; 5,554,499; and 5,747,292. Accordingly, this invention also provides the processes for obtaining the proteins and polypeptides of this invention as well as the products obtainable and obtained by these processes.

The proteins and polypeptides also can be obtained by chemical synthesis using a commercially available automated peptide synthesizer such as those manufactured by Perkin Elmer/Applied Biosystems, Inc., Model 430A or 431A, Foster City, Calif. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook et al., supra, using the host cell and vector systems. This invention further provides a process for producing the receptors and ligands identified by this invention, an analog, a mutein or a fragment thereof, by growing a host cell containing a nucleic acid molecule encoding for these products wherein the nucleic acid is operatively linked to a promoter of RNA transcription. The host cell is grown under suitable conditions such that the nucleic acid is transcribed and translated into protein and purifying the product so produced.

Also provided by this application are these products described herein conjugated to a detectable agent for use in diagnostic methods. For example, detectably labeled proteins and polypeptides containing the receptor or alternatively the ligand can be bound to a solid support as defined above and used for the detection and purification of the binding partner. They also are useful as immunogens for the production of antibodies. The proteins and fragments of this invention are useful in an in vitro assay system to screen for agents or drugs which either inhibit or augment the cytokine pathways and biological effects and to test possible therapies.

Assay Methods or Screens

In the first step of the method, a cell or “test cell” consists of the gene coding for the mutated receptor inserted and expressed in a suitable host cell. Eukaryotic cells can be used for the yeast two-hybrid screen and eucaryotic cells, are preferred for the biological screening and in clinical use.

After the putative receptor-ligand pair or test cells expressing them are constructed, the functional screening assay is run to determine if the putative pair has the required biological activity. The first preliminary screen is a variation of the yeast two-hybrid screen which identifies receptor-ligand binding pairs. Any cell which can express a foreign gene, such as a prokaryotic cell (bacterial such as E. coli) or a eukaryotic cell, is a suitable recipient cell for the practice of this invention. Eukaryotic cells, such as a yeast cells, animal cells, e.g., murine, rat, simian or a human cell, e.g., a human tumor cell, can be transduced with one or more genes coding for the mutated receptor or cytokine. A more detailed description of this in vitro screen is provided below. The other screen identifies biological activity, i.e, it determines whether the ligand specifically binds to and activates the mutant receptor. T-cells isolated from a subject or cultured T cell lines are preferred recipient cells for use in this assay. Established T cell lines are commercially available from sources such as the American Type Culture Collection (ATCC), 10801 University Blvd. Manassas, Va. 20110-2209, U.S.A., or they can be constructed by transducing a recipient cell with a gene coding for the mutant receptor and culturing the cells under conditions that favor replication of the transduced gene in cell progeny. Tumor-infiltrating lymphocytes are the most preferred test cells for this invention because they invade tumors and can be grown from tumor samples using the cytokine IL-2. Using the methods summarized below, a recipient cell is transduced with the gene under conditions favoring expression of the mutated receptor on the surface of the cell. The recipient cell is then contacted with an effective amount of the mutated ligand partner, in an amount effective to induce the biological response associated with the binding of the wild-type receptor to its wild-type ligand. In a preferred embodiment, the mutated receptor-ligand binding pair is the IL-2 receptor binding pair. The biological response associated with wild-type receptor-ligand binding is growth and proliferation of TIL in response to foreign antigens. The ability of the mutated receptor to support proliferation of the activated CTL is readily demonstrated by methods known in the art. For example, activated cell lines that express the mutated or chimeric receptor can be tested for growth in the absence of the wild-type cytokine. A separate control is concurrently conducted wherein the TIL receive an effective amount of wild-type cytokine.

The mutated cytokine binding partner used to activate the receptor is then can be administered by means of ex vivo or in vivo gene therapy.

In each of the above, instances, a modification of the polymerase chain reaction (as provided below) and Northern analysis can be conducted prior to performing the screen to ensure replication and expression of the transduced genes. Monoclonal antibodies also can be raised against the mutated receptor and/or cytokine and used in ELISA to determine replication and expression of the transduced gene by the recipient or host cell.

Animal Models

Prior to use in the clinic, the inventions described herein are assayed in an animal model. To minimize difficulty in interpreting results from animal models, the receptor-ligand pairs should be tested in the organism from which the ligand was derived. This will minimize complications arising from immune rejection of the modified receptor-expressing cells (cellular and humoral) as well as immune rejection (humoral) of the administered ligand. Unfortunately, to avoid these problems it would be necessary to construct a mutant receptor-ligand pair from, for example a mouse, in order to test the system in a mouse model. It should be possible to generate CTL suitable for use in the invention, perhaps in the context of the mouse B16 melanoma model. Utilizing this system, the model can comprise the following steps:
a) generating anti-B16 CTL from these mice;
b) transducing the mice with the mutant IL-2 receptor;
c) administering the transduced CTL into the mice; and
d) administering the mutant ligand.

The assay would measure rejection of B16 cells in either a pretreatment or active treatment setting. In the active treatment setting, the mice would have been pre-exposed to B16 tumor cells prior to receptor-ligand therapy, and in the pretreatment setting, the mice would be challenged with B16 cells at some point after initiation of receptor-ligand therapy.

It would not be desirable to test a human receptor-ligand pair in a fully immunocompetent mouse. However, it may be desirable to test the human receptor-ligand pairs in the human peripheral blood lymphocyte-severe combined immunodeficiency mouse (Hu-PBL-SCID) (described in Tary-Lehmann et al. (1995) Immunol. Today 16(11):529-33 and available from Jackson Labs, Bar Harbor, Me.) or the Hu-PBL-SCID-Beige mouse model (described in McBride et al. (1995) J. Med. Virol. 47(2):130-38 and available from Tacomic, Germantown, N.V.). SCID mice lack mature B and T lymphocytes and can be reconstituted with human PBLs. SCID/Beige mice have deficient NK cell activity in addition to their lack of B and T lymphocytes.

The scope of this invention encompasses any receptor-ligand pair. The following examples specifically describe one embodiment of this invention. Accordingly, the scope of the invention is not to be limited to the following example.

Interleukin-2 Receptor/Ligand Pairs

Early structure-function studies on IL-2 were difficult to interpret due to the multimeric nature of its receptor. A substantial body of research has illuminated the mechanism by which IL-2 interacts with its receptor and initiates a signal transduction cascade that leads to proliferation in cells of lymphoid origin. The availability of cell lines expressing the individual IL-2 receptor subunits has allowed detailed analysis of receptor/ligand interactions. The IL-2 receptor is a heterotrimeric complex consisting of alpha (55 kDa), beta (75 kDa) and gamma (64 kDa) subunits. These proteins can be differentially combined to form receptor complexes with vying affinities for IL-2 as follows:

TABLE 1

| RECEPTOR SUBUNIT | AFFINITY | DISSOC. CONSTANT (Kd) |
|---|---|---|
| α, β, γ | High | 10⁻¹¹ |
| α, β | Pseudo high* | 10⁻¹⁰ |
| β, γ | Intermediate | 10⁻⁹ |
| α | Low | 10⁻⁸ |

*Binds IL-2 but does not transmit the growth signal

IL-2 toxicity originates from secondary responses mediated by cells expressing IL-2 receptors rather than direct toxic effects of the IL-2 protein itself In fact, human (“hIL-2”) analogs have been identified containing point mutations (Ala38 and Lys42) that allow activation of the intermediate affinity receptor but not the high affinity receptor and result in lower level induction of IL-1β and TNF-α, and lower toxicity, as compared to native hIL-2 (see EP 0673257).

A functional receptor complex (i.e., capable of transmitting a growth signal) requires the presence of the β and γ chains. However, when species-specific preferential IL-2-binding is observed, it is the a subunit that confers species-specific recognition of IL-2 by the high affinity receptor complex. Liu et al. (1996) Cytokine 8:613-21, have shown that murine lymphoid cells genetically modified to express hIL-2Rα, hIL-2Rβ, murine IL-2 receptor subunit γ (“mILR-2γ”), proliferate in response to low dose hIL-2 while both hIL-2 and mIL-2 induce proliferation of a cell line expressing mILR-2α, hILR-2β, mILR-2γ. This data combined with the fact that hIL-2 can bind to both the mIL-2R and hIL-2R complexes, but hIL-2 does not bind to the hIL-2R receptor complex suggests that the IL-2Rα subunit determines the ligand-binding species specificity.

In one embodiment of this invention, chimeric receptors are utilized. For example, the human and mouse IL-2 receptors share extensive sequence homology, yet the mouse IL-2 does not interact with the human IL-2 receptor. Mouse-human chimeric α and/or β receptor subunits can be constructed that have the ability to react with mIL-2 (via the mIL-2 receptor extracellular domain) and transmit the-IL-2 proliferative signal (via the hIL-2R intracellular domain). TIL that express this construct would respond to mlL-2 and could be specifically stimulated in vivo, presumably without associated hIL-2-like toxicity in humans.

U.S. Pat. No. 5,747,292, (Greenberg) discloses the use of chimeric receptor pairs that are distinct from the chimeric pairs of this invention. The chimeric receptor constructs covered by the Greenberg patent consist of the intracellular portion of the IL-2 receptor (a chain) fused to the extracellular domain of a heterologous receptor. For example, Greenberg proposes fusing the extracellular domain of the GM-CSF receptor to the intracellular domain of the IL2α chain receptor in a lymphoid cell to obtain the physiologic effects of IL-2 when the cell is exposed to GM-CSF. By this method, Greenberg suggests that the beneficial effects of IL-2 can be obtained without the toxicity associated with IL-2 therapy. However, the constructs and methods disclosed in the Greenberg patent merely substitute one cytokine toxicity for another (i.e., GM-CSF is toxic when given systemically in high doses). Also, it is not clear that the modified IL-2 receptor will retain complete functionality upon interaction with the heterologous ligand. Although a compelling case is made that at least some of the IL-2 receptor functionality is maintained, there is no guarantee that fusions of other heterologous extracellular (ligand-binding) domains will perform as expected.

In contrast, the compositions and methods of this invention do not involve any heterologous receptor sequences. The compositions of this invention contain a point mutation (i.e., changing a single amino acid) in the IL-2 molecule (the ligand) that abolishes its ability to bind to the normal IL-2 receptor complex. A “complimenting” point mutation is made in the α or β IL-2 receptor chain that restores a productive interaction with the mutant IL-2. This allows the expression of the mutant receptor in lymphoid cells of interest that are then infused into a patient. A specific immune response is stimulated in vivo by administration of the mutant IL-2 ligand. Since the mutant IL-2 ligand cannot interact with normal IL-2 receptor complexes, toxicity will be avoided. The compositions and methods also increase the specific activity of various therapies by many orders of magnitude.

Previously Characterized IL-2/IL-2 Receptor Mutants

As noted above, the screen of this invention can be used to identify an IL-2 mutant that interacts with a previously characterized IL-2 receptor β mutant, or an IL-2 receptor β library or for a mutant that interacts with a previously characterized IL-2 mutant. Mutant IL-2 will be expressed and secreted but not utilized by the vast majority of the cells in the screen, thus avoiding the problem of background due to productive interaction with wild-type receptors. Another benefit is that purification of active mutant IL-2 protein is not required and any IL-2-dependent cell line can be used for the screen. When screening a mutant IL-2 library for activity with a mutant IL-2Rβ, the method may further comprise blocking the endogenous IL-2β activity first by expressing a gene coding antisense IL-2β because the IL-2 library would contain copies of the wild-type gene. Gene(s) coding for IL-2β antisense are introduced into the cell thereby reducing the production of IL-2β protein. Some previously characterized hIL-2 mutants are listed in the table below:

TABLE 2
Characterized hIL-2 Mutations

| Mutation | Alpha-Binding | Beta-Binding | %-Activity |
|---|---|---|---|
| Lys35→Ala^{a} | − | + | 25 |
| Arg38→Leu^{a} | − | + | 37 |
| Phe42→Lys^{a} | − | + | 2 |
| Lys43→Glu^{a} | − | + | 25 |
| Trp121→Ser^{b} | − | − | 0.15 |
| Cys58→Ser^{b} | − | − | 0.135 |
| leu17→Asn^{b} | − | − | 1.6 |
| Asp20→Lys^{b} | + | − | 0.135 |

^{a}Sauve et al. (1991) PNAS 88: 4636-4640
^{b}Collins et al. (1988) PNAS 85: 7709-7713

One mutant identified is IL-2(Asp20→Lys). This mutant exhibits less than 0.2% of the biologic activity of wild-type IL-2. IL-2 (Asp20→Lys) is unable to bind to the IL-2 receptor β subunit of the high affinity receptor complex while retaining wild-type binding affinity for the IL-2Rα subunit. The mutant protein presents with an identical near-UV circular dichroism spectrum as the wild-type IL-2 indicating that this amino acid substitution has no gross effect on overall protein conformation.

Table 3 below identifies several additional IL-2 pairs that are useful in the methods of this invention. The left hand column identifies mutant receptors and the top vertical row identifies various mutant ligands. The sequences identified in Table 3 are provided in Imler et al. (1992) EMBO J. 11(6):2053. As identified in Table 3, pairs IL-2Rβ H1133 paired with mIL-2 (hlL-2) D34H (D20H) and IL-2Rβ H133K paired with mIL-2 (hlL-2) D34H (D20H) are preferred pairs in that the mutant pairs bind with almost equal affinity (6 and 8, respectively) as wild-type (3).

TABLE 3

| | mIL-2 (hIL-2) | mIL-2 (hIL-2) | mIL-2 (hIL-2) |
|---|---|---|---|
| | w+ | D34H (D20H) | 334K (D20K) |
| IL-2Rβ w+ | 3* | 82 | NB |
| IL-2Rβ H133A | 36 | 6 | 36 |
| IL-2Rβ H133D | 53 | 11 | 37 |
| IL-2Rβ H133K | NB | 8 | NT |

NB: Non Binding
NT: Not Tested
mIL-2 = mouse IL-2
hIL-2 = human IL-2
mIL-2 mutant D34H is equivalent to hIL-2 mutant D20H
mIL-2 mutant D34K is equivalent to hIL-2 mutant D20K
Note that hIL-2 (D20K) does not bind to the wild-type IL-2Rβ but binds reasonably well to IL-2Rβ H133A and IL-2Rβ H133D. Also, hIL-2 D20H binds poorly to wild-type IL-2Rβ but has a strong interaction with IL-2Rβ H133A, IL-2Rβ H133K, and IL-2Rβ H133D.

Direct Screening in a Human IL-2-Dependent Cell Line

There are many well characterized IL-2-dependent T cell lines that are available through ATCC. The most studied of these is the mouse line, CTLL-2. This cell line expresses the murine high affinity IL-2 receptor complex that is responsive to hIL-2 and is frequently used to assay the bioactivity of human IL-2 protein preparations. This cell line (or any of several other mouse or human lines) is cotransfected with a IL-2 receptor β mutant library and the IL-2(Asp20→Lys) expressing plasmid. Ideally, these are coexpressed from the same plasmid. Alternatively, a stable G418-selected IL-2(Asp20→Lys)-expressing clonal population can be established prior to introducing the IL-2 receptor β mutant library. In one embodiment a simple cotransfection is conducted with independent IL-2 and IL-2 receptor β plasmids.

Upon expansion of the cultures in the absence of exogenous wild-type IL-2, the library plasmids are recovered or amplified by PCR and sequenced. The identified IL-2 receptor , mutants are then retested in a pure TIL system. Isolation and sequencing of the genes encoding these proteins is then conducted using methods well known in the art.

Yeast Two-Hybrid Screening for IL-2(Asp20→Lys)-Binding IL-2Rβ Mutants

The yeast two-hybrid screening method is technically simple and very rapid such that several millions of library clones can be screened in just a few days. All of the elements of the system are commercially available. In this screen, the β-GAL indicator gene will only be activated if the nuclear localization signal (NLS) and DNA-binding domain (DBD) are brought into physical contact by IL-2 mutant/IL2 mutant receptor interaction and cotransported to the nucleus via the NLS. Although the IL-2 receptor β mutants isolated from this screen will bind to IL-2(Asp20→Lys), it must also be confirmed in a human T cell line. If both of the screening procedures described above are implemented concurrently, mutants obtained by each method can be tested and verified in the other system.

Briefly, the yeast two-hybrid system can be used and constructed as follows. The genes coding for putative ligand or receptor can be obtained by PCR and cloned in-frame, as confirmed by sequencing, into the GAL4 DNA binding domain (GAL4bd) vector pAS1CYH2. A more detailed account of the plasmids used in the procedure for the yeast two-hybrid system can be found in Hu et al. (1994) J. Biol. Chem. 269:30069-30072.

Vectors Useful in Genetic Modifications

Prior to conducting the screens identified above, it is necessary to transduce the appropriate recipient cell with the gene coding for the mutated receptor and/or the cytokine. Many methods of successful in vitro and in vivo gene transfer are available to the skilled artisan. The description provided below is merely a summary of the known methods to illustrate a few embodiments within the scope of this invention.

In general, genetic modifications of cells in vitro, ex vivo and in vivo, employed in the present invention are accomplished by introducing a vector containing a polypeptide or transgene encoding a heterologous or an altered antigen. A variety of different gene transfer vectors, including viral as well as non-viral systems can be used. Viral vectors useful in the genetic modifications of this invention include, but are not limited to adenovirus, adeno-associated virus vectors, retroviral vectors and adeno-retroviral chimeric vectors.

Construction of Recombinant Adenoviral Vectors or Adeno-Associated Virus Vectors

Adenovirus and adeno-associated virus vectors useful in the genetic modifications of this invention may be produced according to methods already taught in the art. (see, e.g., Karlsson et al. (1986) EMBO 5:2377; Carter (1992) Current Opinion in Biotechnology 3:533-539; and Muzcyzka (1992) Current Top. Microbiol. Immunol. 158:97-129; GENE TARGETING: A PRACTICAL APPROACH (1992) ed. A. L. Joyner, Oxford University Press, NY). Several different approaches are feasible. Preferred is the helper-independent replication deficient human adenovirus system.

The recombinant adenoviral vectors based on the human adenovirus 5 (Virology 163:614-617, 1988) are missing essential early genes from the adenoviral genome (usually E1A/E1B), and are therefore unable to replicate unless grown in permissive cell lines that provide the missing gene products in trans. In place of the missing adenoviral genomic sequences, a transgene of interest can be cloned and expressed in cells infected with the replication deficient adenovirus. Although adenovirus-based gene transfer does not result in integration of the transgene into the host genome (less than 0.1% adenovirus-mediated transfections result in transgene incorporation into host DNA), and therefore is not stable, adenoviral vectors can be propagated in high titer and transfect non-replicating cells. Human 293 cells, which are human embryonic kidney cells transformed with adenovirus E1A/E1B genes, typify useful permissive cell lines and are commercially available from the ATCC. However, other cell lines which allow replication-deficient adenoviral vectors to propagate therein can be used, including HeLa cells.

Additional references describing adenovirus vectors and other viral vectors which could be used in the methods of the present invention include the following: Horwitz, M. S., Adenoviridae and Their Replication, in Fields B. et al. (eds.) VIROLOGY, Vol. 2, Raven Press New York, pp. 1679-1721, 1990); Graham F. et al. pp. 109-128 in METHODS IN MOLECULAR BIOLOGY, Vol. 7: GENE TRANSFER AND EXPRESSION PROTOCOLS, Murray, E. (ed.), Humana Press, Clifton, N.J. (1991); Miller N. et al. (1995) FASEB Journal 9:190-199; Schreier H. (1994) Pharmaceutica Acta Helvetiae 68:145-159; Schneider and French (1993) Circulation 88:1937-1942; Curiel D. T. et al. (1992) Human Gene Therapy 3:147-154; Graham F. L. et al., WO 95/00655; Falck-Pedersen E. S., WO 95/16772; Denefle P. et al., WO 95/23867; Haddada H. et al., WO 94/26914; Perricaudet M. et al., WO 95/02697; and Zhang, W. et al., WO 95/25071. A variety of adenovirus plasmids are also available from commercial sources, including, e.g., Microbix Biosystems of Toronto, Ontario (see, e.g., Microbix Product Information Sheet: Plasmids for Adenovirus Vector Construction, 1996). See also, the papers by Vile et al. (1997) Nature Biotechnology 15:840-841; and Feng et al. (1997) Nature Biotechnology, 15:866-870, describing the construction and use of adeno-retroviral chimeric vectors that can be employed for genetic modifications.

Additional references describing AAV vectors which could be used in the methods of the present invention include the following: Carter, B., HANDBOOK OF PARVOVIRUSES, Vol. I, pp. 169-228, 1990; Berns, VIROLOGY, pp. 1743-1764 (Raven Press 1990); Carter B. (1992) Curr. Opin. Biotechnol. 3:533-539; Muzyczka N. (1992) Current Topics in Micro. and Immunol. 158:92-129; Flotte T. R. et al. (1992) Am. J. Respir. Cell Mol. Biol. 7:349-356; Chatterjee et al. (1995) Ann. NY Acad. Sci. 770:79-90; Flotte T. R. et al., WO 95/13365; Trempe J. P. et al., WO 95/13392; Kotin, R. (1994) Human Gene Therapy, 5:793-801; Flotte, T. R. et al. (1995) Gene Therapy 2:357-362; Allen J. M., WO 96/17947; and Du et al. (1996) Gene Therapy 3:254-261.

Construction of Retroviral Vectors

Retroviral vectors useful in the methods of this invention are produced recombinantly by procedures already taught in the art. For example, WO 94/29438 describes the construction of retroviral packaging plasmids and packaging cell lines. As is apparent to the skilled artisan, the retroviral vectors useful in the methods of this invention are capable of infecting the cells described herein. The techniques used to construct vectors, and transfix and infect cells are widely practiced in the art. Examples of retroviral vectors are those derived from murine, avian or primate retroviruses. Retroviral vectors based on the Moloney murine leukemia virus (MOMLV) are the most commonly used because of the availability of retroviral variants that efficiently infect human cells. Other suitable vectors include those based on the Gibbon Ape Leukemia Virus (GALV) or HIV.

In producing retroviral vector constructs derived from the Moloney murine leukemia virus (MoMLV), in most cases, the viral gag, pol and env sequences are removed from the virus, creating room for insertion of foreign DNA sequences. Genes encoded by the foreign DNA are usually expressed under the control of the strong viral promoter in the LTR. Such a construct can be packed into viral particles efficiently if the gag, pol and env functions are provided in trans by a packaging cell line. Thus, when the vector construct is introduced into the packaging cell, the gag-pol and env proteins produced by the cell, assemble with the vector RNA to produce infectious virions that are secreted into the culture medium. The virus thus produced can infect and integrate into the DNA of the target cell, but does not produce infectious viral particles since it is lacking essential packaging sequences. Most of the packaging cell lines currently in use have been transfected with separate plasmids, each containing one of the necessary coding sequences, so that multiple recombination events are necessary before a replication competent virus can be produced. Alternatively, the packaging cell line harbors an integrated provirus. The provirus has been crippled so that, although it produces all the proteins required to assemble infectious viruses, its own RNA cannot be packaged into virus. Instead, RNA produced from the recombinant virus is packaged. The virus stock released from the packaging cells thus contains only recombinant virus.

The range of host cells that may be infected by a retrovirus or retroviral vector is determined by the viral envelope protein. The recombinant virus can be used to infect virtually any other cell type recognized by the env protein provided by the packaging cell, resulting in the integration of the viral genome in the transduced cell and the stable production of the foreign gene product. In general, murine ecotropic env of MoMLV allows infection of rodent cells, whereas amphotropic env allows infection of rodent, avian and some primate cells, including human cells. Amphotropic packaging cell lines for use with MoMLV systems are known in the art and commercially available and include, but are not limited to, PA12 and PA317. Miller et al. (1985) Mol. Cell. Biol. 5:431-437; Miller et al. (1986) Mol. Cell. Biol. 6:2895-2902; and Danos et al. (1988) PNAS (USA) 85:6460-6464. Xenotropic vector systems exist which also allow infection of human cells.

The host range of retroviral vectors has been altered by substituting the env protein of the base virus with that of a second virus. The resulting, “pseudotyped”, virus has the host range of the virus donating the envelope protein and expressed by the packaging cell line. Recently, the G-glycoprotein from vesicular stomatitis virus (VSV-G) has been substituted for the MoMLV env protein. Burns et al. (1993) PNAS (USA) 90:8033-8037; and PCT patent application WO 92/14829. Since infection is not dependent on a specific receptor, VSV-G pseudotyped vectors have a broad host range.

Usually, the vectors will contain at least two heterologous genes or gene sequences: (i) the therapeutic gene to be transferred; and (ii) a marker gene that enables tracking of infected cells. As used herein, “therapeutic gene” can be an entire gene or only the functionally active fragment of the gene capable of compensating for the deficiency in the patient that arises from the defective endogenous gene. Therapeutic gene also encompasses antisense oligonucleotides or genes useful for antisense suppression and ribozymes for ribozyme-mediated therapy. For example, in the present invention, a therapeutic gene may be one that neutralizes an immunosuppressive factor or counters its effects.

Nucleotide sequences for the therapeutic gene will generally be known in the art or can be obtained from various sequence databases such as GenBank. The therapeutic gene itself will generally be available or can be isolated and cloned using the polymerase chain reaction PCR (Perkin-Elmer) and other standard recombinant techniques. The skilled artisan will readily recognize that any therapeutic gene can be excised as a compatible restriction fragment and placed in a vector in such a manner as to allow proper expression of the therapeutic gene in hematopoietic cells.

A marker gene can be included in the vector for the purpose of monitoring successful transduction and for selection of cells into which the DNA has been integrated, as against cells which have not integrated the DNA construct. Various marker genes include, but are not limited to, antibiotic resistance markers, such as resistance to G418 or hygromycin. Less conveniently, negative selection may be used, including, but not limited to, where the marker is the HSV-tk gene, which will make the cells sensitive to cytotoxic agents such as acyclovir and gancyclovir. Alternatively, selections could be accomplished by employment of a stable cell surface marker to select for transgene expressing cells by FACS sorting. The NeoR (neomycin/G418 resistance) gene is commonly used but any convenient marker gene whose sequences are not already present in the recipient cell, can be used.

The viral vector can be modified to incorporate chimeric envelope proteins or nonviral membrane proteins into retroviral particles to improve particle stability and expand the host range or to permit cell type-specific targeting during infection. The production of retroviral vectors that have altered host range is taught, for example, in WO 92/14829 and WO 93/14188. Retroviral vectors that-can target specific cell types in vivo are also taught, for example, in Kasahara et al. (1994) Science 266:1373-1376. Kasahara et al. describe the construction of a Moloney murine leukemia virus (MOMLV) having a chimeric envelope protein consisting of human erythropoietin (EPO) fused with the viral envelope protein. This hybrid virus shows tissue tropism for human red blood progenitor cells that bear the receptor for EPO, and is therefore useful in gene therapy of sickle cell anemia and thalassemia. Retroviral vectors capable of specifically targeting infection of cells are preferred for in vivo gene therapy.

The viral constructs can be prepared in a variety of conventional ways. Numerous vectors are now available which provide the desired features, such as long terminal repeats, marker genes, and restriction sites, which may be further modified by techniques known in the art. The constructs may encode a signal peptide sequence to ensure that cell surface or secreted proteins encoded by genes are properly processed post-translationally and expressed on the cell surface if appropriate. Preferably, the foreign gene(s) is under the control of a cell specific promoter.

Expression of the transferred gene can be controlled in a variety of ways depending on the purpose of gene transfer and the desired effect. Thus, the introduced gene may be put under the control of a promoter that will cause the gene to be expressed constitutively, only under specific physiologic conditions, or in particular cell types.

The retroviral LTR (long terminal repeat) is active in most hematopoietic cells in vivo and will generally be relied upon for transcription of the inserted sequences and their constitutive expression (Ohashi et al. (1992) PNAS 89:11332; and Correll et al. (1992) Blood 80:331). Other suitable promoters include the human cytomegalovirus (CMV) immediate early promoter and the U3 region promoter of the Moloney Murine Sarcoma Virus (MMSV), Rous Sarcoma Virus (RSV) or Spleen Focus Forming Virus (SFFV).

Examples of promoters that may be used to cause expression of the introduced sequence in specific cell types include Granzyme A for expression in T-cells and NK cells, the CD34 promoter for expression in stem and progenitor cells, the CD8 promoter for expression in cytotoxic T-cells, and the CD11b promoter for expression in myeloid cells.

Inducible promoters may be used for gene expression under certain physiologic conditions. For example, an electrophile response element may be used to induce expression of a chemoresistance gene in response to electrophilic molecules. The therapeutic benefit may be further increased by targeting the gene product to the appropriate cellular location, for example the nucleus, by attaching the appropriate localizing sequences.

The vector construct is introduced into a packaging cell line which will generate infectious virions. Packaging cell lines capable of generating high titers of replication-defective recombinant viruses are known in the art, see for example, WO 94/29438. Viral particles are harvested from the cell supernatant and purified for in vivo infection using methods known in the art such as by filtration of supernatants 48 hours post transfection. The viral titer is determined by infection of a constant number of appropriate cells (depending on the retrovirus) with titrations of viral supernatants. The transduction efficiency can be assayed 48 hours later by a variety of methods, including Southern blotting.

After viral transduction, the presence of the viral vector in the transduced cells or their progeny can be verified such as by PCR. PCR can be performed to detect the marker gene or other virally transduced sequences. Generally, periodic blood samples are taken and PCR conveniently performed using e.g. NeoR probes if the NeoR gene is used as marker. The presence of virally transduced sequences in bone marrow cells or mature hematopoietic cells is evidence of successful reconstitution by the transduced cells. PCR techniques and reagents are well known in the art, see, generally, PCR PROTOCOLS, A GUIDE TO METHODS AND APPLICATIONS. Innis, Gelfand, Sninsky & White, eds. (Academic Press, Inc., San Diego, 1990) and commercially available (Perkin-Elmer).

Non-viral vectors, such as plasmid vectors useful in the genetic modifications of this invention, can be produced according to methods taught in the art. References describing the construction of non-viral vectors include the following: Ledley F D (1995) Human Gene Therapy 6:1129-1144; Miller N. et al. (1995) FASEB Journal 9:190-199; Chonn A. et al. (1995) Curr. Opin. in Biotech. 6:698-708; Schofield J P et al. (1995) British Med. Bull. 51: 56-71; Brigham K. L. et al. (1993) J. Liposome Res. 3:31-49; Brigham K. L., WO 91/06309; Felgner P. L. et al., WO 91/17424; Solodin et al. (1995) Biochemistry 34:13537-13544; WO 93/19768; Debs et al., WO 93/25673; Felgner P. L. et al. U.S. Pat. No. 5,264,618; Epand R. M. et al., U.S. Pat. No. 5,283,185; Gebeyehu et al., U.S. Pat. No. 5,334,761; FeIgner P. L. et al., U.S. Pat. 5,459,127; Overell R. W. et al., WO 95/28494; Jessee, WO 95/02698; Haces and Ciccarone, WO 95/17373; and Lin et al., WO 96/01840.

Therapeutic Applications

In one embodiment, the agents identified herein as effective for their intended purpose can be administered to subjects having tumors or cancer. When the agent is administered to a subject such as a mouse, a rat or a human patient, the agent can be added to a pharmaceutically acceptable carrier and systemically or topically administered to the subject. To determine patients that can be beneficially treated, tumor regression can be assayed. Therapeutic amounts can be empirically determined and will vary with the pathology being treated, the subject being treated and the efficacy and toxicity of the therapy. When delivered to an animal, the method is useful to further confirm efficacy of the agent. As an example of an animal model, groups of nude mice (Balb/c NCR nulnu female, Simonsen, Gilroy, CA) are each subcutaneously inoculated with about 10⁵ to about 10⁹ hyperproliferative, cancer cells as defined herein. When the tumor is established, the cells expressing the mutated receptor is administered, for example, by subcutaneous injection around the tumor. The mutated cytokine ligand or a gene expressing the ligand is then administered in an effective amount. Tumor measurements to determine reduction of tumor size are made in two dimensions using venier calipers twice a week. Other animal models may also be employed as appropriate.

Administration in vivo can be effected in one dose, continuously or intermittently throughout the course of treatment. Methods of determining the most effective means and dosage of administration are well known to those of skill in the art and will vary with the composition used for therapy, the purpose of the therapy, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

More particularly, an agent of the present invention also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

It is to be understood that while the invention has been described in conjunction with the above embodiments, that the foregoing description and the following examples are intended to illustrate and not limit the scope of the invention. For example, any of the above-noted compositions and/or methods can be combined with known therapies or compositions. Other aspects, advantages and modifications within the scope of the invention will be apparent to those skilled in the art to which the invention pertains.

1. A method for identifying novel receptor-ligand binding pairs, comprising contacting a cell expressing a mutated receptor with a putative ligand and assaying for receptor-ligand binding and biological response and binding of higher affinity as compared to the corresponding wild-type receptor.

2. The method of claim 1, wherein the ligand is a cytokine.

3. The method of claim 2, wherein the mutated receptor selected from the group consisting of mutated IL-2, mutated G-CSF, mutated TNF-α, and mutated IFN-γ receptor.

4. The method of claim 1, wherein the putative ligand is selected from the group consisting of IL-2, G-CSF, TNF-α and IFN-γ.

5. The method of claim 1, wherein the ligand is the corresponding mutated ligand.

6. The method of claim 1, further comprising purifying the mutated receptor.

7. The method of claim 1, further comprising purifying the mutated ligand.

8. The method of claim 6, further comprising isolation and determining the sequence of the receptor.

9. The method of claim 7, further comprising isolation and determining the sequence of the ligand.

10. A method of activating a receptor comprising administering to a subject an effective amount of a host cell expressing a polynucleotide encoding a mutated receptor and an effective amount of a ligand that selectively binds to the receptor wherein the receptor is activated by the binding of said ligand to said receptor.

11. The method of claim 10, wherein the ligand is a cytokine.

12. The method of claim 11, wherein the cytokine is selected from the group consisting of IL-2, G-CSF, TNF-α and IFN-γ.

13. The method of claim 10, wherein the host cell is a tumor infiltrating lymphocyte or a hematopoietic stem cell.

14. The method of claim 10, wherein the polynucleotide comprises a sequence coding for an IL-2 β chain and a sequence coding for an IL-2 α chain.

15. The method of claim 10, further comprising administering an effective amount of a co-stimulatory molecule to the subject.

16. The method of claim 10, wherein the cell is a hematopoietic stem cell and the polynucleotide encodes mutated G-CSF.

17. A method of inducing an immune response in a subject bearing a tumor comprising the steps of:
administering to said tumor an effective amount of a polynucleotide encoding a mutated IL-2 receptor and
activating said mutated IL-2 receptor by means of locoregionally administering a mutated ligand that selectively binds to the mutant receptor.

18. The method of claim 17, further comprising administering an effective amount of a co-stimulatory molecule to the subject.

19. The method claim 18, wherein the polynucleotide is administered via a viral vector that is directly injected into the tumor of the subject.

20. The method claim 15 or 18, wherein the co-stimulatory molecule is administered as a polynucleotide encoding the molecule.

21. A polynucleotide encoding a mutated receptor identified by the method of claim 1.

22. A polynucleotide encoding a mutated ligand identified by the method of claim 1.

23. A method for selectively activating cytokine expression in a subject comprising the steps of: administering to said subject an effective amount of a host cell(s) expressing a polynucleotide encoding a mutated receptor and administering an effective amount of a ligand that selectively binds to said mutated receptor activating cytokine expression in said host cell(s).

24. The method of claim 14, wherein the IL-2 β chain sequence encodes for IL-2 β H133A and the ligand is selected from the group consisting of mIL-2 334K, hIL-2 D20K, mIL-2 D34H, or hIL-2 D20H.

25. The method of claim 14, wherein the IL-2 β chain sequence encodes for IL-2 β H133D and the ligand is selected from the group consisting of mIL-2 334K, hIL-2 D20K, mIL-2 D34H, or hIL-2 D20H.

26. The method of claim 14, wherein the IL-2 β chain sequence encodes for IL-2 β H133K and the ligand is selected from the group consisting of mIL-2 334K, hIL-2 D20K, mIL-2 D34H, or hIL-2 D20H.

27. A method for identifying mutant IL-2 receptor-ligand binding pairs, comprising:
1) introducing a plurality of polynucleotides encoding for IL-2 receptor β mutants into an IL-2 dependent cell that expresses a mutant IL-2, and
2) selecting said cells that survive in the absence of IL-2, and
3) from said surviving cells, recovering the polynucleotides encoding for said IL-2 receptor β mutants, wherein said mutant IL-2 and said recovered IL-2 receptor β mutant bind each other and provide the biological response of the wild-type IL-2 ligand-receptor pair.

28. A method for identifying mutant IL-2 receptor-ligand binding pairs, comprising:
1) introducing a plurality of polynucleotides encoding for IL-2 mutants into an IL-2 dependent cell that expresses a mutant IL-2 receptor β, and
2) selecting said cells that survive in the absence of IL-2, and
3) from said surviving cells, recovering the polynucleotides encoding for said IL-2 receptor β mutants, wherein said mutant IL-2 and said recovered IL-2 receptor β mutant bind each other and provide the biological response of the wild-type IL-2 ligand-receptor pair.

29. The method of claim 2, wherein endogenous IL-2 receptor β activity is blocked during said selection step.

30. A method for identifying mutant IL-2 receptor-ligand binding pairs, comprising:
1) providing cells comprising a reporter gene operatively linked to an upstream activating sequence recognized by a selected transcription factor, and expressing an IL-2 mutant fused to the DNA binding domain of said transcription factor and,
2) introducing a plurality of polynucleotides encoding for IL-2 receptor β mutants fused to the activation domain of said transcription factors into said cell and,
3) selecting the cells that then express said reporter gene, and
4) recovering the polynucleotides encoding for said IL-2 receptor β mutant fusion proteins from said expressing cells,
wherein said recovered IL-2 receptor β mutant interacts with the mutant IL-2 ligand.

31. A method for identifying mutant IL-2 receptor-ligand binding pairs, comprising:
1) providing cells comprising a reporter gene operatively linked to an upstream activating sequence recognized by a selected transcription factor, (need something here?,) and expressing an IL-2 receptor β mutant fused to the DNA binding domain of said transcription factor and,
2) introducing a plurality of polynucleotides encoding for IL-2 mutants fused to the activation domain of said transcription factors into said cell and,
3) selecting the cells that then express said reporter gene, and
4) recovering the polynucleotides encoding for said IL-2 mutant fusion proteins from said expressing cells, wherein said recovered IL-2 receptor βmutant interacts with the mutant IL-2 ligand.

32. The method of claim 1, wherein said expressed mutant IL-2 is a human IL-2 selected from the group consisting of human IL-2 with lysine35 mutated to alanine, human IL-2 with arginine38 mutated to leucine, human IL-2 with phenylalanine42 mutated to lysine, human IL-2 with lysine43 mutated to glutamine, human IL-2 with tryptophan121 mutated to serine, human IL-2 with cysteine58 mutated to serine, human IL-2 with leucine17 mutated to asparagine, or human IL-2 with aspartic acid20 mutated to lysine.

33. The method of claim 2, wherein said expressed mutant IL-2 receptor β is selected from the group consisting of IL-2 receptor β H133A, IL-2 receptor β H133D, or IL-2 receptor β H133K.

34. The method of claim 1, further comprising isolation and determining the sequence of the recovered IL-2 receptor β mutant.

35. The method of claim 2, further comprising determining the sequence of the recovered IL-2 mutant.

36. The method of claim 4, further comprising determining the sequence of the recovered IL-2 receptor β mutant.

37. The method of claim 5, further comprising determining the sequence of the recovered IL-2 mutant.

38. The method of claim 23, wherein the polynucleotide comprises a sequence coding for an IL-2 β H133A chain and a sequence coding for an IL-2 α chain and wherein the ligand is selected from the group consisting of mIL-2 334K, hIL-2 D20K, mIL-2 D34H, or hIL-2 D20H.

39. The method of claim 23, wherein the polynucleotide comprises a sequence coding for an for IL-2 β H133D and a sequence coding for an IL-2 α chain and wherein the ligand is selected from the group consisting of mIL-2 334K, hIL-2 D20K, mIL-2 D34H, or hIL-2 D20H.

40. The method of claim 23, wherein the polynucleotide comprises a sequence coding for an for IL-2 p H1133K and a sequence coding for an IL-2 a chain and wherein the ligand is selected from the group consisting of mIL-2 334K, hIL-2 D20K, mIL-2 D34H, or hIL-2 D20H.
